(19) United States
(12) Patent Application Publication
(10) Pub. No.: US 2025/0345600 A1
(43) Pub. Date: Nov. 13, 2025

(54) COMPLEX HANDPIECE EQUIPPED WITH MICRONEEDLES AND SKIN CARE DEVICE INCLUDING THE SAME

(71) Applicant: WEERO Co., Ltd., Anyang-si (KR)

(72) Inventor: Dong Shin LEE, Hwaseong-si (KR)

(21) Appl. No.: 19/094,051

(22) Filed: Mar. 28, 2025

(30) Foreign Application Priority Data
May 9, 2024 (KR) 10-2024-0061106

(51) Int. Cl. A61N 1/06; A61B 5/01; A61N 1/05; A61N 1/32

(52) U.S. Cl. CPC A61N 1/06; A61B 5/01; A61N 1/0502; A61N 1/328

(57) ABSTRACT

Proposed is a complex handpiece. The handpiece includes a housing and a needle assembly. The housing has an internal space and a plurality of holes formed at a front face thereof. The needle assembly is located in the internal space of the housing, and includes a plurality of microneedles for applying a high-frequency current. The microneedles are movable in a longitudinal direction of the housing such that tips of the microneedles are exposed to an outside of the housing through the holes. The microneedles become longer as being positioned from an outermost edge of the front face of the housing to a central portion of the front face of the housing such that the tips of the microneedles are arranged convexly with the central portion as an apex. The complex handpiece can uniformly insert the microneedles into the skin and perform both microneedle high-frequency treatment and ultrasonic treatment.

### CROSS REFERENCE TO RELATED APPLICATION

The present application claims priority to Korean Patent Application No. 10-2024-0061106 filed on May 9, 2024, the entire contents of which are incorporated herein for all purposes by this reference.

### BACKGROUND

### Technical Field

The disclosure relates to a handpiece and a skin care device including the same. In particular, the disclosure relates to a complex handpiece including microneedles to be inserted into the skin and apply a high-frequency current, and a skin care device including the complex handpiece.

### Description of Related Technology

Recently, as interest in skin has increased, various devices related to skin care are being developed. One of widely known skin care techniques is microneedle high-frequency treatment, which is a combination of microneedle treatment and typical fractional laser treatment. This is a method of inserting very fine needles into the skin to create a treatment column and also applying a high-frequency current.

### SUMMARY

One aspect is a complex handpiece that can uniformly insert a plurality of microneedles into the skin, and a skin care device including the same.

Another aspect is a complex handpiece that can perform both microneedle high-frequency treatment and ultrasonic treatment, and a skin care device including the same.

Another aspect is a complex handpiece that includes a housing and a needle assembly. The housing has an internal space and a plurality of holes formed at a front face thereof. The needle assembly is located in the internal space of the housing, and includes a plurality of microneedles for applying a high-frequency current. The microneedles are formed to be movable in a longitudinal direction of the housing such that tips of the microneedles are exposed to an outside of the housing through the plurality of holes. The microneedles become longer as being positioned from an outermost edge of the front face of the housing to a central portion of the front face of the housing such that the tips of the microneedles are arranged convexly with the central portion as an apex.

Among the plurality of microneedles, a height difference between the tip of a central microneedle arranged in the central portion of the housing and the tip of an outermost microneedle arranged in the outermost edge of the housing may range from 0.5 to 1 mm.

The plurality of microneedles may be arranged in one of a hexagonal pattern, a triangular pattern, and a square pattern.

The complex handpiece may further include an ultrasonic unit. The ultrasonic unit is positioned on the front side of the housing to be spaced apart from the needle assembly, and alternately or simultaneously outputs first and second ultrasonic energies having different frequencies at predetermined time intervals.

The plurality of microneedles may be evenly arranged around the ultrasonic unit.

The plurality of microneedles may be arranged to surround the ultrasonic unit or arranged at both sides of the ultrasonic unit.

At least one of the plurality of microneedles may be replaced with a needle-shaped temperature sensor that detects skin temperature.

Another aspect is a skin care device that includes a main body and a handpiece. The main body includes a high-frequency generator that generates a high-frequency current. The handpiece is combined with the main body, and includes a plurality of microneedles that apply the high-frequency current generated by the high-frequency generator to a skin.

According to the handpiece and the skin care device of the disclosure, the tips of the plurality of microneedles are arranged convexly with the center as the apex, so that the plurality of microneedles can be uniformly inserted into the skin.

In addition, according to the handpiece and the skin care device of the disclosure, the microneedles that are inserted into the skin and apply a high-frequency current and the ultrasonic unit that alternately or simultaneously outputs ultrasonic energy having different frequencies to the skin are arranged to be spaced apart from each other, so that both microneedle high-frequency treatment and ultrasonic treatment are possible with one handpiece.

### BRIEF DESCRIPTION OF THE DRAWINGS

FIG. 1 is a block diagram showing a skin care device according to an embodiment of the disclosure.

FIG. 2 is a cross-sectional view showing a complex handpiece according to a comparative example.

FIG. 3 is a cross-sectional view showing a complex handpiece according to the first embodiment of the disclosure.

FIG. 4 is a plan view showing a front face of a complex handpiece according to the first embodiment of the disclosure.

FIG. 5 is a plan view showing a front face of a complex handpiece according to the second embodiment of the disclosure.

FIG. 6 is a cross-sectional view showing a complex handpiece according to the second embodiment of the disclosure.

FIG. 7 is a plan view showing a front face of a complex handpiece according to the third embodiment of the disclosure.

### DETAILED DESCRIPTION

Generally, when a high-frequency current is applied to the human body, the molecules that make up the human tissues rub against each other whenever the direction of the current changes, and deep heat is generated. Unlike other types of current, a high-frequency current has the advantage of not stimulating sensory or motor nerves and heating specific areas within the human tissue without causing muscle contraction.

When a high-frequency current is applied to the skin through microneedles, deep heat promotes collagen formation in the dermis layer and regenerates muscle fibers destroyed due to aging, improving skin elasticity. In addition, deep heat can melt hardened sebum in the epidermis layer with a sense of heat, resulting in a pore purification effect.

As a related art, Korean Patent No. 10-2117711 discloses an RF high-frequency generator that is implemented to deliver strong RF energy after inserting microneedles of a small size into the dermal layer to manage problematic skin such as reducing wrinkles and improving elasticity by stimulating fibroblasts and collagen fibers. In particular, the RF high-frequency generator disclosed in Korean Patent No. 10-2117711 does not cause the difference in height between the tips of the microneedles through automatic alignment to improve the height difference of the microneedles that occurs when the tips of the microneedles are lifted due to the surface tension of the solder during soldering.

However, if there is no height difference at the tips of the microneedles, the skin may be pressed concavely by the pressure applied to the skin just before the multiple microneedles penetrate the skin. At this time, there is a problem that among the multiple microneedles, some microneedles positioned at the edge can be inserted into the skin to a sufficient depth, whereas some microneedles positioned at the center are not inserted into the concavely pressed skin or are inserted shallowly, making it difficult to accurately apply a high-frequency current to the target area.

Therefore, there is a need to develop a skin care device that can evenly insert the multiple microneedles into the skin.

Another skin treatment technique is the use of ultrasound treatment, which rapidly crosses two different wavelengths of ultrasound to produce a new effect through the instantaneous contraction-relaxation action of skin connective tissue that cannot be created with the existing single-frequency ultrasound.

Typically, for the microneedle high-frequency treatment and the ultrasound treatment, different devices are used respectively. Thus, there is a problem that in order to use both treatments, both a skin care device and an ultrasound device are required.

Hereinafter, various embodiments of the disclosure will be described in detail with reference to the accompanying drawings. The disclosure may, however, be embodied in many different forms and should not be construed as being limited to the embodiments set forth herein. Rather, these embodiments are provided so that the disclosure will be thorough and complete and will fully convey the scope of the disclosure to those skilled in the art.

In the following description of embodiments, techniques that are well known in the art and not directly related to the disclosure are not described. This is to clearly convey the subject matter of the disclosure by omitting an unnecessary explanation. In the disclosure, the same or corresponding elements are denoted by the same reference numerals.

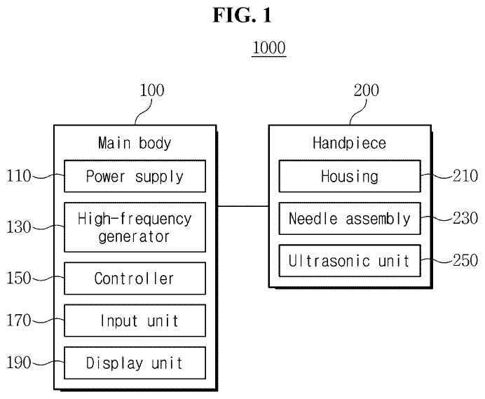

FIG. 1 is a block diagram showing a skin care device according to an embodiment of the disclosure.

Referring to FIG. 1, a skin care device 1000 according to an embodiment of the disclosure includes a main body 100 and a complex handpiece 200. The main body 100 includes a high-frequency generator 130 that generates a high-frequency current. The complex handpiece 200 is combined with the main body 100 and includes a plurality of microneedles 231 that apply a high-frequency current generated by the high-frequency generator 130 to the skin.

The main body 100 may include a power supply 110, the high-frequency generator 130, a controller 150, an input unit 170, and a display unit 190.

The power supply 110 can apply power to the high-frequency generator 130 to generate a high-frequency current. To this end, the power supply 110 may be directly connected to an external power source through a power cable or the like, or may include a battery.

The high-frequency generator 130 can generate a high-frequency current through power supplied from the power supply 110, and is electrically connected to the microneedles 231 to enable the microneedles 231 to apply a high-frequency current to the skin. The high-frequency generator 130 may generate a high-frequency current having a frequency of 0.1 to 10 MHZ, but is not limited thereto.

The controller 150 controls the overall operation and signals of the skin care device 1000. In response to a signal inputted from the input unit 170, the controller 150 may control whether to apply power to the high-frequency generator 130, and control the intensity, time, pattern, etc. of the high-frequency current generated from the high-frequency generator 130.

The input unit 170 can receive various kinds of information such as numeric information and character information, and transmit input signals in relation to setting or controlling various functions of the skin care device 1000 to the controller 150. The input unit 170 may be composed of at least one of a keypad and a touchpad that create input signals in response to a user's touch or manipulation. In particular, the input unit 170 may create input signals for operating the skin care device 1000 and receive input data for controlling the intensity, time, patterns, etc. of a high-frequency current from the user.

The display unit 190 displays information on a series of operation states, operation results, etc. that occur during the function execution of the skin care device 1000. The display unit 190 may be composed of a liquid crystal display (LCD), a thin film transistor-liquid crystal display (TFT-LCD), an organic light-emitting diode (OLED), a flexible display, a three-dimensional (3D) display, etc. If the display unit 190 is formed of a touch screen, it may perform some or all of the functions of the input unit 170. In particular, the display unit 190 may output data on the intensity, time, pattern, etc. of a high-frequency current being outputted.

The complex handpiece 200 may include a plurality of microneedles 231 that are combined with the main body 100 and apply a high-frequency current generated by the high-frequency generator 130 to the skin. When a high-frequency current is applied to the skin through the microneedles 231, molecules constituting the skin tissue rub against each other and thus deep heat is generated. The deep heat can promote collagen formation in the dermis layer and regenerate muscle fibers destroyed due to aging, thereby improving skin elasticity. In addition, the deep heat can melt hardened sebum in the epidermis layer with a sense of heat, thereby obtaining a pore purification effect.

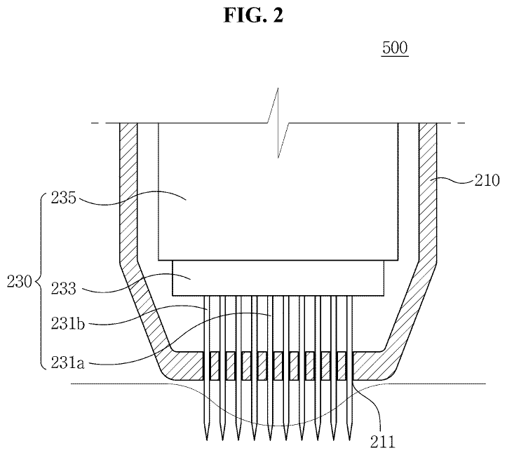

FIG. 2 is a cross-sectional view showing a complex handpiece according to a comparative example.

Referring to FIG. 2, if there is no height difference at the tips of the microneedles 231 arranged in the complex handpiece 500, the skin may be pressed into a concave shape due to the pressure applied to the skin just before the plurality of microneedles 231 penetrate the skin. As a result, the microneedles 231 arranged at the outermost edge among the plurality of microneedles 231 can be inserted into the skin to a sufficient depth, whereas the microneedles 231 arranged in a central portion are not inserted into the concavely pressed skin or are inserted shallowly, making it difficult to accurately apply the high-frequency current to the target area.

To solve this problem, the complex handpiece 200 according to the disclosure has an enhanced arrangement of the plurality of microneedles 231 in which the tips of the microneedles 231 protrude convexly based on a central portion of the arrangement, thereby enabling the plurality of microneedles 231 to be uniformly inserted into the skin.

Hereinafter, with reference to the attached drawings, the complex handpiece 200 according to embodiments of the disclosure will be described in more detail.

### First Embodiment

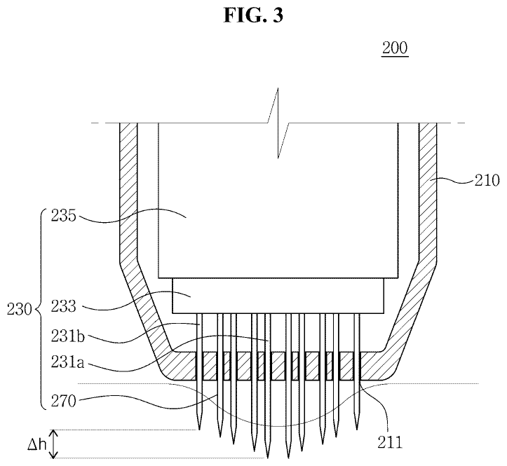

FIG. 3 is a cross-sectional view showing a complex handpiece according to the first embodiment of the disclosure.

Referring to FIG. 3 together with FIG. 1, the complex handpiece 200 includes a housing 210 and a needle assembly 230. The needle assembly 230 is positioned inside the housing 210 and includes the plurality of microneedles 231 for applying a high-frequency current.

The housing 210 is combined with the main body 100 and may have a cylindrical shape of a certain length so that it can be held with the hand. The housing 210 has an internal space, and a plurality of holes 211 are formed at a front face of the housing 210. The holes 211 are provided to allow the microneedles 231 to penetrate and may be formed corresponding to the number and positions of the microneedles 231.

The needle assembly 230 is located in the internal space of the housing 210 and is detachably combined with the housing 210 to be replaceable. Specifically, the needle assembly 230 may include the plurality of microneedles 231, a fixing plate 233 for fixing the microneedles 231, and an actuator 235 for moving the fixing plate 233.

The microneedle 231 has a sharp tip to be inserted into the dermal layer of the skin and can apply a high-frequency current. The tip of the microneedle 231 is formed of a metal material to allow a high-frequency current to flow, and the remaining part other than the tip is formed of an insulating material to prevent a high-frequency current from flowing to a site other than the target site. The diameter of the microneedle 231 may be 10 to 50 μm, but is not limited thereto.

The fixing plate 233 is located in the internal space of the housing 210 and fixes the microneedles 231 arranged at regular intervals. In addition, the fixing plate 233 can prevent damage such as bending or breaking of the microneedles 231 that penetrate the skin and are inserted into the dermal layer.

In particular, on the fixing plate 233, the microneedles 231 that become longer as they are positioned from the outermost edge of the front face of the housing 210 to the central portion are arranged, so that the tips of the microneedles 231 can be arranged convexly with the central portion of the front face of the housing 210 as the apex.

In this case, a height difference (Δh) between the tip of the central microneedle 231a arranged in the central portion of the housing 210 and the tip of the outermost microneedle 231b arranged in the outermost edge of the housing 210 may range from 0.5 to 1 mm.

If the height difference between the tip of the central microneedle 231a and the tip of the outermost microneedle 231b is less than 0.5 mm, the skin may be pressed into a concave shape by the pressure applied to the skin just before the plurality of microneedles 231 penetrate the skin, as shown in FIG. 2. As a result, the outermost microneedle 231b among the plurality of microneedles 231 can be inserted into the skin to a sufficient depth, whereas the central microneedle 231a may not be inserted into the concavely pressed skin or may be inserted shallowly.

In addition, if the height difference between the tip of the central microneedle 231a and the tip of the outermost microneedle 231b exceeds 1 mm, the central microneedle 231a can be inserted into the skin to a sufficient depth, but the outermost microneedle 231b may not be inserted into the skin to a sufficient depth. Therefore, it is preferable that the height difference between the tip of the central microneedle 231a and the tip of the outermost microneedle 231b ranges from 0.5 to 1 mm.

Accordingly, the needle assembly 230 of the complex handpiece 200 according to the first embodiment can enable the plurality of microneedles 231 to be inserted into the skin evenly even when the skin is pressed concavely.

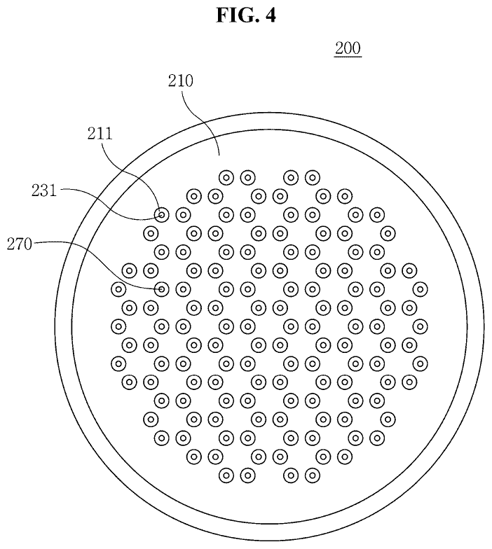

The plurality of microneedles 231 may be arranged in a hexagonal pattern as shown in FIG. 4. Alternatively, the plurality of microneedles 231 may be arranged in other patterns, such as a triangular pattern, a square pattern, etc.

FIG. 4 is a plan view showing a front face of a complex handpiece according to the first embodiment of the disclosure.

Referring to FIG. 4, the plurality of microneedles 231 may be arranged in a hexagonal pattern. When arranging the plurality of microneedles 231 at the same interval in a given area, arranging the microneedles 231 in a hexagonal pattern allows for arranging more microneedles 231. In addition, the complex handpiece 200 may form a bipolar configuration in which the microneedles 231 that apply a positive current and the microneedles 231 that apply a negative current are arranged alternately.

Additionally, in the complex handpiece 200 according to the first embodiment, at least one of the plurality of microneedles 231 may be replaced with a needle-shaped temperature sensor 270 that detects skin temperature, as shown in FIGS. 3 and 4. Since the high-frequency current applied from the microneedle 231 generates deep heat in the skin, the controller (150 in FIG. 1) can prevent the skin from overheating by stopping the application of the high-frequency current when the skin temperature detected by the temperature sensor 270 is higher than a predetermined temperature.

The actuator 235 can move the microneedles 231 in the longitudinal direction of the housing 210, and thus the tips of the microneedles 231 can be exposed to the outside of the housing 210 through the plurality of holes 211 of the housing 210. In particular, the actuator 235 can control the degree to which the microneedles 231 are exposed to the outside of the housing 210, thereby adjusting the depth at which the microneedles 231 are inserted into the skin. The actuator 235 may use a hydraulic or pneumatic cylinder, but is not limited thereto.

### Second Embodiment

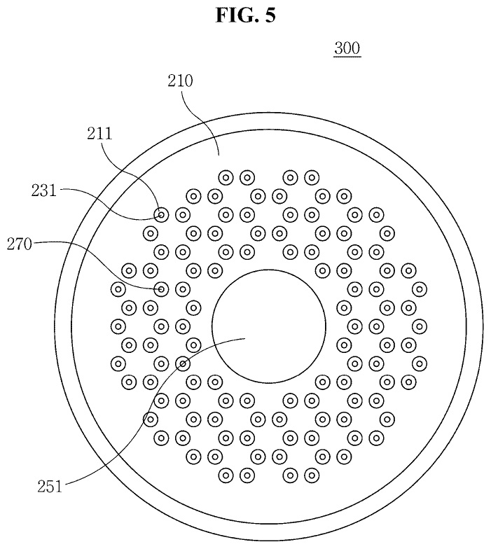

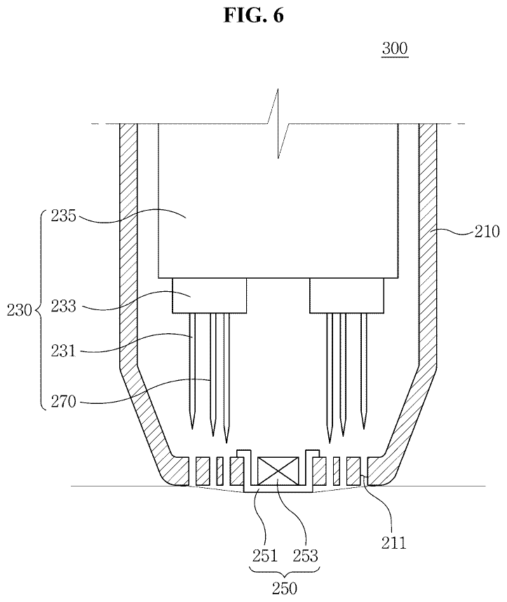

FIG. 5 is a plan view showing a front face of a complex handpiece according to the second embodiment of the disclosure, and FIG. 6 is a cross-sectional view showing a complex handpiece according to the second embodiment of the disclosure.

Referring to FIGS. 5 and 6, the complex handpiece 300 according to the second embodiment further includes an ultrasonic unit 250. The ultrasonic unit 250 is positioned at the central portion of the front face of the housing 210, and the microneedles 231 of the needle assembly 230 are arranged to surround the ultrasonic unit 250 on the front face of the housing 210.

In the complex handpiece 300 according to the second embodiment, the microneedles 231 of the needle assembly 230 are inserted into the skin and apply a high-frequency current, and the ultrasonic unit 250 alternately or simultaneously outputs ultrasonic energy having different frequencies to the skin. The microneedles 231 of the needle assembly 230 and the ultrasonic unit 250 are disposed to be spaced apart from each other, thereby enabling both the microneedle high-frequency treatment and the ultrasonic treatment in one complex handpiece 300.

The housing 210, the needle assembly 230, and the temperature sensor 270 of the complex handpiece 300 according to the second embodiment are the same as those of the complex handpiece 200 according to the first embodiment, so a description thereof will be omitted.

The ultrasonic unit 250 can alternately output or simultaneously output ultrasonic energy having different frequencies at predetermined time intervals. The ultrasonic unit 250 may include an ultrasonic terminal 251 that contacts the skin, and a vibrator 253 that vibrates the ultrasonic terminal 251.

The ultrasonic terminal 251 can be in contact with the skin and transmit vibrations generated from the ultrasonic terminal 251 to the skin. The ultrasonic terminal 251 may be made of a metal material including at least one of aluminum, aluminum alloy, stainless steel, titanium, and titanium alloy, but is not limited thereto. In addition, one or more ultrasonic terminals 251 may be used, and the material and thickness of the ultrasonic terminal 251 may be set in consideration of the resonant frequency.

The vibrator 253 may be a piezoelectric ceramic element. For example, the vibrator 253 may output first ultrasonic energy having a frequency of 1 to 3 MHz and second ultrasonic energy having a frequency of 3 to 20 MHz.

Ultrasound with a frequency of 1 to 3 MHz can pass through the epidermis and dermis layers and reach the subcutaneous fat layer. Therefore, ultrasound with a frequency of 1 to 3 MHz has the effect of lifting the skin by breaking down fat in the subcutaneous fat layer or contracting and relaxing muscles.

Ultrasound with a frequency of 3 to 20 MHz corresponds to a frequency with an amplitude smaller than the thickness of the cell wall, and can reach the smallest particles within the cell. Therefore, ultrasound with a frequency of 3 to 20 MHz can vibrate cells in the epidermis and dermis layers to circulate lymph, and can affect the activity of enzymes such as matrix metalloproteinases (MMPs) and heat shock proteins (HSPs) involved in skin aging.

MMPs break down damaged collagen, elastin, etc. so that new tissues can take root. However, if MMPs are secreted excessively, the skin barrier may be damaged or inflammation may occur. HSPs are proteins involved in cell regeneration and immune capabilities. Ultrasound having a frequency of 3 to 20 MHz can suppress the secretion of MMPs and promote the secretion of HSPs, thereby achieving skin regeneration and immune enhancement effects. In addition, ultrasound having a frequency of 3 to 20 MHz can promote the production of hyaluronic acid, which stores moisture and supplies moisture to cells when necessary, thereby achieving the effects of improving dryness and moisture.

The ultrasonic unit 250 may include at least one vibrator 253. For example, one vibrator 253 may output both the first and second ultrasonic energies, or two vibrators 253 may output the first and second ultrasonic energies, respectively and separately.

Additionally, the ultrasonic unit 250 may alternately or simultaneously output the first and second ultrasonic energies having different frequencies at certain time intervals under the control of the controller (150 in FIG. 1).

### Third Embodiment

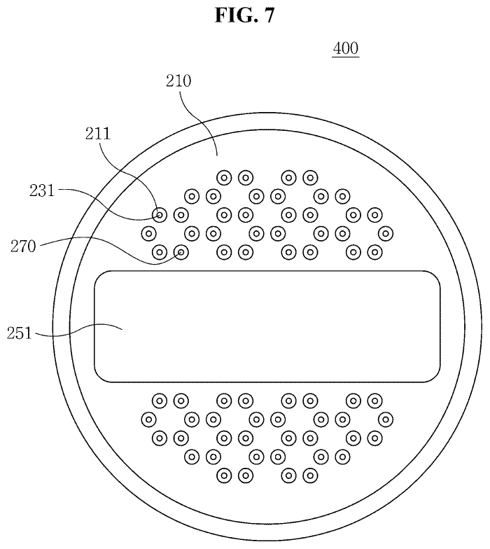

FIG. 7 is a plan view showing a front face of a complex handpiece according to the third embodiment of the disclosure.

Referring to FIG. 7, in the complex handpiece 400 according to the third embodiment, the ultrasonic unit 250 is positioned at the central portion of the front face of the housing 210, and the microneedles 231 of the needle assembly 230 are arranged at both sides (e.g., upper and lower sides, as shown) of the ultrasonic unit 250 rather than arranged to surround the ultrasonic unit 250.

The housing 210, the needle assembly 230, the ultrasonic unit 250, and the temperature sensor 270 of the complex handpiece 400 according to the third embodiment are the same as those of the complex handpiece 300 according to the second embodiment, so a description thereof will be omitted.

According to the disclosure, the arrangement of the needle assembly 230 and the ultrasonic unit 250 is not particularly limited as long as they are arranged spaced apart from each other at the front face of the housing 210.

While the disclosure has been particularly shown and described with reference to exemplary embodiments thereof, it will be understood by those skilled in the art that various changes in form and details may be made therein without departing from the scope of the disclosure as defined by the appended claims.

What is claimed is:

1. A complex handpiece comprising:
a housing comprising an internal space and a plurality of holes formed at a front face thereof; and
a needle assembly located in the internal space of the housing, and including a plurality of microneedles configured to apply a high-frequency current, the microneedles movable in a longitudinal direction of the housing such that tips of the microneedles are exposed to an outside of the housing through the plurality of holes,
the microneedles configured to become longer as being positioned from an outermost edge of the front face of the housing to a central portion of the front face of the housing such that the tips of the microneedles are arranged convexly with the central portion as an apex.

2. The complex handpiece of claim 1, wherein among the plurality of microneedles, a height difference between the tip of a central microneedle arranged in the central portion of the housing and the tip of an outermost microneedle arranged in the outermost edge of the housing ranges from 0.5 mm to 1 mm.

3. The complex handpiece of claim 1, wherein the plurality of microneedles are arranged in one of a hexagonal pattern, a triangular pattern, or a square pattern.

4. The complex handpiece of claim 1, further comprising:
an ultrasonic unit positioned on the front side of the housing to be spaced apart from the needle assembly, and configured to alternately or simultaneously output first and second ultrasonic energies having different frequencies at predetermined time intervals.

5. The complex handpiece of claim 4, wherein the plurality of microneedles are evenly arranged around the ultrasonic unit.

6. The complex handpiece of claim 4, wherein the plurality of microneedles are arranged to surround the ultrasonic unit or arranged at both sides of the ultrasonic unit.

7. The complex handpiece of claim 1, wherein at least one of the plurality of microneedles is replaced with a needle-shaped temperature sensor configured to detect skin temperature.

8. A skin care device comprising:
a main body including a high-frequency generator configured to generate a high-frequency current; and
a handpiece combined with the main body, and including a plurality of microneedles configured to apply the high-frequency current generated by the high-frequency generator to a skin,
the handpiece comprising:
a housing combined with the main body, and comprising an internal space and a plurality of holes formed at a front face thereof, and
a needle assembly located in the internal space of the housing, and
including the plurality of microneedles,
the microneedles movable in a longitudinal direction of the housing such that tips of the microneedles are exposed to an outside of the housing through the plurality of holes,
the microneedles configured to become longer as being positioned from an outermost edge of the front face of the housing to a central portion of the front face of the housing such that the tips of the microneedles are arranged convexly with the central portion as an apex.
